(19) United States
(12) Patent Application Publication
(10) Pub. No.: US 2005/0009919 A1
(43) Pub. Date: Jan. 13, 2005

(54) Treating cachexia and excessive catabolism with (-)-hydroxycitric acid

(76) Inventor: Dallas L. Clouatre, Santa Monica, CA (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 10/616,321

(22) Filed: Jul. 7, 2003

(51) Int. Cl. A61K 31/19; A61K 31/366

(52) U.S. Cl. 514/574; 514/460

(57) ABSTRACT

The inventor has discovered that (−)-hydroxycitric acid (including the forms of its various salts) is useful for treating and ameliorating cachexia, health-threatening catabolism and unhealthful weight loss, such as is characteristic of sarcopenia. The dosage will depend on factors such as the starting weight of the individual and the percentage of the calories in the diet derived from fats. On a 30 percent fat diet, an efficacious daily dosage for most individuals will be between 250 mg and 3 grams per day. It may prove beneficial to deliver the desired dosage only once per day, preferably prior to the noon meal. The weight-gain effects of HCA are compromised by the actions compounds such as caffeine and ephedrine, hence these should be avoided. Due to the biphasic characteristics of HCA, there is an obvious overlap between dosages that can lead to weight gain and the higher dosages that can lead to weight loss in those who are above their ideal body weights. There is little or no evidence that HCA ingested even in quite large amounts causes significant weight loss in individuals who are at or below their idea weights or exhibit a body mass index (BMI) at or below 20. It is to be expected that dosage will need to be matched to the current state of a given individual suffering from cachexia, catabolism or sarcopenia.

### BACKGROUND OF THE INVENTION

1. Field Of The Invention

This invention relates to pharmaceutical compositions containing (−)-hydroxycitric acid useful for treating and ameliorating cachexia, health-threatening catabolism and unhealthful weight loss.

2. Description Of Prior Art

Cachexia is defined as a state of general wasting. It is caused by a variety of factors, for instance anorexia, illness such as infections or cancer, poor alimentary habits, and disturbances in digestion and nutrient absorption linked to damage to the digestive tract. These factors lead to the progressive loss of weight, lipid store and muscular body mass along with a negative nitrogen balance with a clinically significant depletion of circulating and visceral proteins. Aside from instances of frank starvation and semi-starvation, cachexia is typical of patients with liver, kidney and gastrointestinal tract diseases, cancers, severe trauma and HIV/AIDS. Based on the anthropometric tables of the Metropolitan Life Insurance Company, one accepted criterion, cachexia may be diagnosed either by a weight loss of more than 10% of the ideal or usual weight or a body weight that falls below the 15th percentile for persons of the same age and height. Some authorities suspect cachexia in cancer patients when weight loss equal to or greater than 5% of pre-morbid weight takes place within a 6 month period.

A large number of different factors have been blamed for cachexia. Most generally agreed upon is the role of cytokines. In HIV patients, tumor necrosis factor-alpha (TNF-α) is viewed as being a significant factor in cachexia. Even in HIV patients whose weight has been stabilized via highly active antiretroviral therapy, loss of lean body mass is common and is driven by catabolic cytokines rather than by inadequate dietary intake. (Roubenoff R, et al. Role of cytokines and testosterone in regulating lean body mass and resting energy expenditure in HIV-infected men. Am J Physiol Endocrinol Metab. July 2002; 283(1):E138-45.) In some forms of cancer cachexia, interleukin-6 (IL-6) may be the more active cytokine involved in cachexia, or at least can be modified by anti-inflammatory treatment with fish oil to reverse weight loss. This treatment also improves the elevated ratio of cortisol-to-insulin. (Barber MD, et al. Effect of a fish oil-enriched nutritional supplement on metabolic mediators in patients with pancreatic cancer cachexia. Nutr Cancer. 2001;40(2):118-24.) Similarly, inflammation and an under-regulation of cytokines appear to be important contributing vectors to tissue loss in sarcopenia, that is, muscle loss with advancing age. (Roubenoff R. Catabolism of aging: is it an inflammatory process? Curr Opin Clin Nutr Metab Care. May 2003; 6(3):295-9.)

Recent work has increased the range of factors involved in cachexia and health-threatening catabolism to include a number of central mechanisms. Some of these factors exhibit peculiar contradictory roles. For instance, cortisol and other counter-regulatory hormones are sources of clearly negative actions in excess catabolism, yet because of the dearth of good pharmacological options, corticosteroids are nevertheless held up as possible treatment modalities. (Inui A. Cancer anorexia-cachexia syndrome: current issues in research and management. CA Cancer J Clin. March-April 2002; 52(2):72-91.) More generally, cachexia is initiated and sustained via a cascade of neurohormonal monoaminergic and other mediators in the central nervous system, including the hypothalamus, which is normally taken as the seat of satiety and appetite. Among the most detailed mediators studied are corticotropin-releasing factor and serotonin which, via the hypothalamic-pituitary-adrenal axis and the sympathetic and parasympathetic nervous systems, stimulate, in turn, the counter-regulatory catecholamines, cortisol, glucagon, etc. while inhibiting anabolic hormones. (Nandi J, et al. Central mechanisms involved with catabolism. Curr Opin Clin Nutr Metab Care. July 2002; 5(4):407-18.) Serotonin, in particular, has proven to be a particularly important vector for appetite suppression and muscle catabolism. Pharmacological inhibition of serotonin synthesis and activity has shown encouraging results. (Laviano A, et al. Neurochemical mechanisms for cancer anorexia. Nutrition. January 2002; 18(1):100-5.) Serotonin, of course, is well-known as acting as a pro-inflammatory under a variety of circumstances. Finally, yet another consideration is the fact that although some have pointed to the suppression of anabolic hormones, such as insulin, as a component in cachexia, elevated plasma insulin levels actually appear to play a role in cytokine-induced anorexia. (Sato T, et al. Involvement of plasma leptin, insulin and free tryptophan in cytokine-induced anorexia. Clin Nutr. April 2003; 22(2):139-46.)

Various measures currently are advocated for the treatment of cachexia. Amongst these are antiserotonergic drugs, gastroprokinetic agents, branched-chain amino acids, eicosapentanoic acid, cannabinoids, melatonin, and thalidomide. Other than the branched-chain amino acids and eicosapentanoic acid, most of these have obvious drawbacks. Even supposedly innocuous agents, such as melatonin, can increase serotonin production and exert pro-inflammatory effects. Such side effects are usually played down—they may seem small in comparison with the alternative, but that does not mean that they do not exist. Quite typical of the drug, as opposed to nutritional approaches is dronabinol (Marinol/Roxane). Among the serious side effects are: hallucinations, severe mood changes, irritability, and euphoria Common side effects are: dizziness, drowsiness, poor coordination, and trouble thinking. Less common are: depression, anxiety, nervousness, headache, hallucinations, blurred vision, rapid heartbeat, frequent or difficult urination, convulsions, and dry mouth. (http://www.wholehealthmd.com/refshelf/drugs) According to conventional wisdom and the published literature, the actions of (−)-hydroxycitric acid would make the compound completely unsuited for use in cachexia. Indeed, it would be expected that the compound might make matters worse rather than better. (−)-Hydroxycitric acid (abbreviated herein as HCA), a naturally-ocurring substance found chiefly in fruits of the species of Garcinia, and several synthetic derivatives of citric acid have been investigated extensively in regard to their ability to inhibit the production of fatty acids from carbohydrates, to suppress appetite, and to inhibit weight gain. (Sullivan AC, Triscari J. Metabolic regulation as a control for lipid disorders. I. Influence of (−)-hydroxycitrrate on experimentally induced obesity in the rodent. American Journal of Clinical Nutrition 1977;30:767.)

Weight loss benefits were first ascribed to HCA, its salts and its lactone in U.S. Pat. No. 3,764,692 granted to John M. Lowenstein in 1973. The claimed mechanisms of action for HCA, most of which were originally put forth by researchers at the pharmaceutical firm of Hoffmann-La Roche, have been summarized in at least two United States patents. In U.S. Pat. No. 5,626,849 these mechanisms are given as follows: “(−) HCA reduces the conversion of carbohydrate calories into fats. It does this by inhibiting the actions of ATP-citrate lyase, the enzyme which converts citrate into fatty acids and cholesterol in the primary pathway of fat synthesis in the body. The actions of (−) HCA increase the production and storage of glycogen (which is found in the liver, small intestine and muscles of mammals) while reducing both appetite and weight gain. (−) Hydroxycitric acid also causes calories to be burned in an energy cycle similar to thermogenesis . . . (−) HCA also increases the clearance of LDL cholesterol . . . . “U.S. Pat. No. 5,783,603 further argues that HCA serves to disinhibit the metabolic breakdown and oxidation of stored fat for fuel via its effects upon the compound malonyl CoA and that gluconeogenesis takes place as a result of this action. The position that HCA acts to unleash fatty acid oxidation by negating the effects of malonyl CoA with gluconeogenesis as a consequence (McCarty MF. Promotion of hepatic lipid oxidation and gluconeogenesis as a strategy for appetite control. Medical Hypotheses 1994;42:215-225) is maintained in U.S. Pat. No. 5,914,326.

Heretofore, HCA has not been suggested in any published literature to be a compound with the ability to prevent weight loss when used appropriately. Although it has been noted by critics of HCA that the compound has not always lived up to its billing as a weight loss agent, no one has realized that the opposite trend is significant, that is, that HCA can cause weight gain. Although overlooked prior to this point, there is considerable evidence in the work of others to support just such a claim. For instance, in recent thorough clinical trials, HCA not only has failed to produce appetite suppression, but actually has led to not-statistically-significant trends toward weight elevation. (Heymsfield SB, et al. Garcinia cambogia (hydroxycitric acid) as a potential antiobesity agent: a randomized controlled trial. JAMA. 1998;280:1596-1600; also Mattes RD, Bormann L. Effects of (−)-hydroxycitric acid on appetitive variables. Physiol Behav. October 2000 1;71(1-2):87-94.)

At least one mechanism by which HCA might lead to weight gain can be found in the prior art, yet no one prior to the present inventor has drawn the appropriate conclusions nor performed the appropriate tests for validation. Albeit they did not pursue the matter, two Roche researchers in 1977 showed that HCA in the cytosol of the cell will activate acetyl CoA carboxylase similarly to the citrate that HCA resembles. (Triscari J, Sullivan AC. Comparative effects of (−)-hydroxycitrate and (+)-allo-hydroxycitrate on acetyl CoA carboxylase and fatty acid and cholesterol synthesis in vivo. Lipids April 1977;12(4): 357-363.)

Not a single one of the patents which have been granted to date for the employment of HCA as an antiobesity agent (U.S. Pat. Nos. 3,764,692; 5,626,849; 5,783,603; 5,914,326 and others proposing the use of HCA as an adjunctive ingredient) has indicated any awareness of the paradoxical biphasic effect of HCA depending upon dosage levels and macronutrient content of the diet. The most recent academic review on the topic, one written by authors associated with the primary producer of HCA products, indicates no awareness of these properties of HCA. (Ohia SE, et al. Safety and mechanism of appetite suppression by a novel hydroxycitric acid extract (HCA-SX). Mol Cell Biochem. September 2002; 238(1-2):89-103.) Neither is any awareness shown in the most recently filed published application on HCA. (U.S. patent application 2003/0119913 A1) Hence, there can be no argument but that the claims of the present inventor are, indeed, novel.

Animal trials conducted by the present inventor have confirmed the weight gain effect of HCA when the compound is delivered at an inadequate dosage level or in an inappropriate manner of intake, especially in conjunction with a diet containing nontrivial amounts of fats. This information was first presented in the inventor's now issued U.S. Pat. No. 6,476,071. Certainly, the typical American dietary pattern in which calories derived from fats account for at least 30% of the total caloric intake is sufficient to cause supplementation with HCA to upregulate acetyl CoA carboxylase and, subsequently, the synthesis of fatty acids from acetyl CoA. The present authors's animal experiments have confirmed that some HCA salts sold commercially and utilized for failed clinical trials will cause weight gain in animals under experimental conditions in which fats account for 30% of ingested calories. These commercial salts appear to suffer from poor assimilation and/or there is some problem with the nature of the compound which is delivered into the body inasmuch as even the so-called solubilized salts which are mixtures of potassium and calcium fail to reduce weight gain in animal experiments. In contradistinction to these commercial HCA products, the same amount of HCA delivered from a fairly good quality potassium salt under the same conditions, as indicated above and discussed below under Preferred Embodiments, inhibited weight gain in this animal experiment. The present inventor's quite surprising discovery, therefore, is that HCA, a putative weight loss compound, with appropriate diet and usage actually leads to weight gain rather than weight loss. Moreover, it does so in a fashion that does not aggravate factors that are important in cases of cachexia, advancing years, and so forth. HCA, for instance, lowers the base rate of glucocorticoids and insulin (by the present inventor, U.S. Pat. No. 6,476,071), and therefore does not aggravate these hormonal pathways that are already disturbed in cases of cachexia and sarcopenia.

### SUMMARY OF THE INVENTION

The inventor has discovered that HCA is useful for treating and ameliorating cachexia, health-threatening catabolism and unhealthful weight loss. Consistency in effect and dosage suggests that HCA be delivered in the form of its potassium or sodium salts. However, other forms of the compound also are efficacious. The dosage will depend on factors such as the starting weight of the individual and the percentage of the calories in the diet derived from fats and/or alcohol. On a 30 percent fat diet, an efficacious daily dosage for most individuals will be between 250 mg and 3 grams per day. It may prove beneficial to deliver the desired dosage only once per day, preferably prior to the noon meal. The weight-gain effects of HCA are compromised by the actions compounds such as caffeine and ephedrine, hence these should be avoided. Due to the biphasic characteristics of HCA, there is an obvious overlap between dosages that can lead to weight gain and the higher dosages that can lead to weight loss in those who are overweight. It should be noted that there is little or no evidence that HCA ingested even in quite large amounts causes significant weight loss in individuals who are at or below their idea weights or exhibit a body mass index (BMI) at or below 20. For maximal benefit, it is to be expected that dosage will need to be matched to the current state of a given individual suffering from cachexia, catabolism or sarcopenia. In the latter case, it is likely that the higher dosages of HCA more typically employed for other health purposes-up to 10 grams or more per day-to control glucocorticoids, etc., may still be used without negative effect inasmuch as, again, available evidence indicates that HCA does not induce weight loss in individuals who are at or below their ideal body weight.

Objects and Advantages

It is an objective of the present invention to provide a method for treating and ameliorating cachexia, health-threatening catabolism and unhealthful weight loss, including in sarcopenia It is a further object of the present invention to provide a means of treating or ameliorating such disorders without inducing or supporting further hormonal and metabolic dysregulation, such as is characteristics of these states. It is yet a further advantage of the present invention to provide a means—one which is accompanied by no side effects-of maintaining proper metabolic functioning and energy expenditure as well as stabilizing weight without resort to invasive medications or special diets. Knowledge of the present invention has the advantage of allowing the use of forms of (−)-hydroxycitric acid, including especially through controlled release formulations, as adjuvants to drugs designed to stabilize or improve long term energy balance.

### DESCRIPTION OF THE PREFERRED EMBODIMENTS

The free acid form and various salts of (−)-hydroxycitric acid (calcium, magnesium, potassium, sodium and mixtures of these) have been available commercially for several years. Any of these materials can be used to fulfill the invention revealed here, but with varying degrees of success. These materials are generally useful in this descending order of efficacy: potassium salt, sodium salt, free acid, magnesium salt, calcium salt. A novel method for improving the efficacy and workability of these forms is provided in that application. Exact dosing will depend upon the form of HCA used, the weight of the individual involved, and the other components of the diet. The previously patented hydroxycitric acid derivatives (mostly amides and esters of hydroxycititric acid, the patents for which are now expired) likely are roughly equivalent to the HCA sodium salt in efficacy and can be applied as hypotensive agents at taught herein by one skilled in the art. Hydroxycitric acid in its free acid form and in its lactone form may be less desirable for long term use due to the ability of these forms to chelate minerals and thereby perhaps lead to mineral loss.

### EXAMPLE 1

### The Biphasic Qualities of (−)-Hydroxycitric Acid

The published literature on HCA gives evidence of both temporal and dosage biphasic effects, albeit very little is made of these. No patent granted on the use of HCA to date makes mention of either effect. Indeed, the weight loss or anti-obesity claims of prior HCA patents would seem to rest largely or even entirely upon the observed appetite-suppressing effects of HCA, and these effects seem to disappear within seven weeks. (Sullivan AC, Triscari J. Metabolic regulation as a control for lipid disorders. I. Influence of (−)-hydroxycitrrate on experimentally induced obesity in the rodent. American Journal of Clinical Nutrition 1977;30:767.) No previous patent on HCA mentions the problematic use of the compound in conjunction with diets which contain significant amounts of fat. Hence the dosage levels commonly suggested, such as in the patent of Hastings, et al. (U.S. Pat. No. 5,626,849), which patent never tested its claims in either animals or humans, will lead to elevated rates of production of fats and to either null results or even weight gain under most human dietary practices in which fats contribute at least 30% of calories. Recent negative clinical results using the amounts of HCA commonly suggested, including in the issued U.S. patents, bear out the truth of this observation. Trials using 1.2 grams to 3 grams of HCA per day derived from calcium (−)-hydroxycitrate and ingested in divided doses before meals have failed to produce anorectic results.

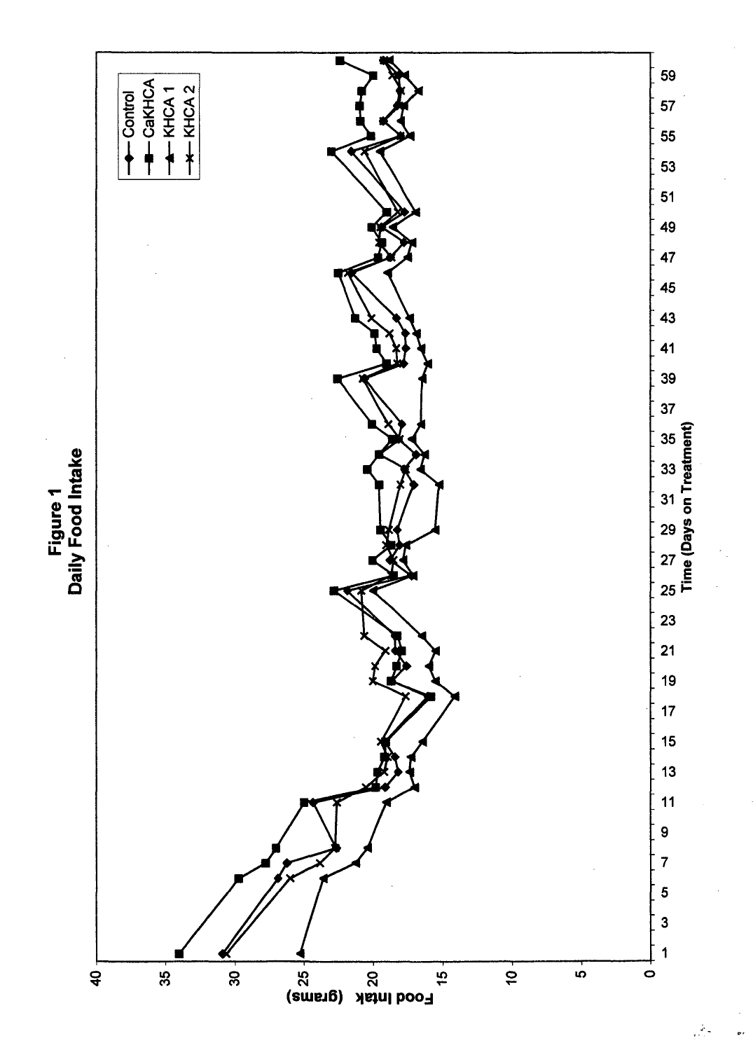

To test the properties of HCA in various forms under conditions similar to those found in human clinical trials, the inventor arranged for male OM rats aged 10 weeks to be fed a diet in which 30% of the calories were obtained from fat under standard conditions. The rats were intubated twice daily with one of three HCA salts or placebo. The amount of HCA in each arm of 5 animals was the minimum dosage which had been found effective in the form of the pure trisodium salt of HCA in tests by Hoffmann-La Roche in animals ingesting a 70% glucose diet, i.e., 0.33 mmoles/kg body weight HCA given twice per day. The HCA salts used were these: CaKHCA=a mixed calcium and potassium HCA salt commercially marketed as being entirely water soluble; KHCA 1=a relatively clean, but still hardly pure potassium salt of HCA with a good mineral ligand attachment supplying 4467 mg potassium/100 grams of material; KHCA 2=an impure potassium salt of HCA with large amounts of gums attached and poor mineral ligand attachment supplying 2169 mg potassium/100 grams of material. FIGS. 1, 2 and 3 summarize the findings.

FIG. 1 shows the change in food intake over the 60 day period of the trial. Initial exposure to the highly palatable fatty diet after being raised on standard rat chow led to a period of elevated food intake in these rats which diminished for all arms over a period of roughly two weeks. Food intake at virtually all points was higher with the CaKHCA than in any other arm. Food intake was initially and remained largely the same in the control and KHCA 2 arms for the length of the trial. Food intake was markedly suppressed in the KHCA 1 arm for the first 25 days and remained quite noticeably depressed in comparison with the other arms for roughly seven weeks. During the last week of the study, however, food intake was only marginally less in the KHCA 1 arm than in the control and KHCA 2 arms despite the lighter average weight of the animals in the KHCA 1 arm. The finding that the appetite suppression from the HCA diminished markedly by the end of the seventh week is in agreement with published data from Roche's animal trials, trials which used synthesized, hence pure HCA, typically in the form of the trisodium salt. The finding that the better quality KHCA 1 salt suppressed appetite and weight gain at this level of intake on a fatty diet rather than a 70% glucose diet indicates that the potassium salt is more active than is the sodium salt of HCA.

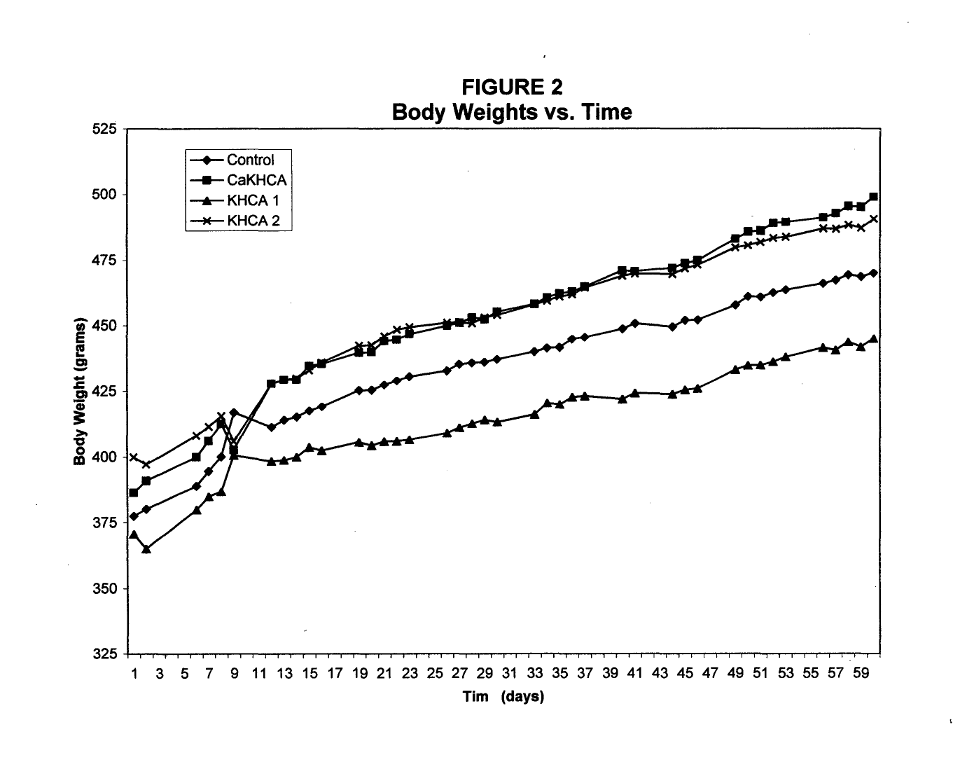

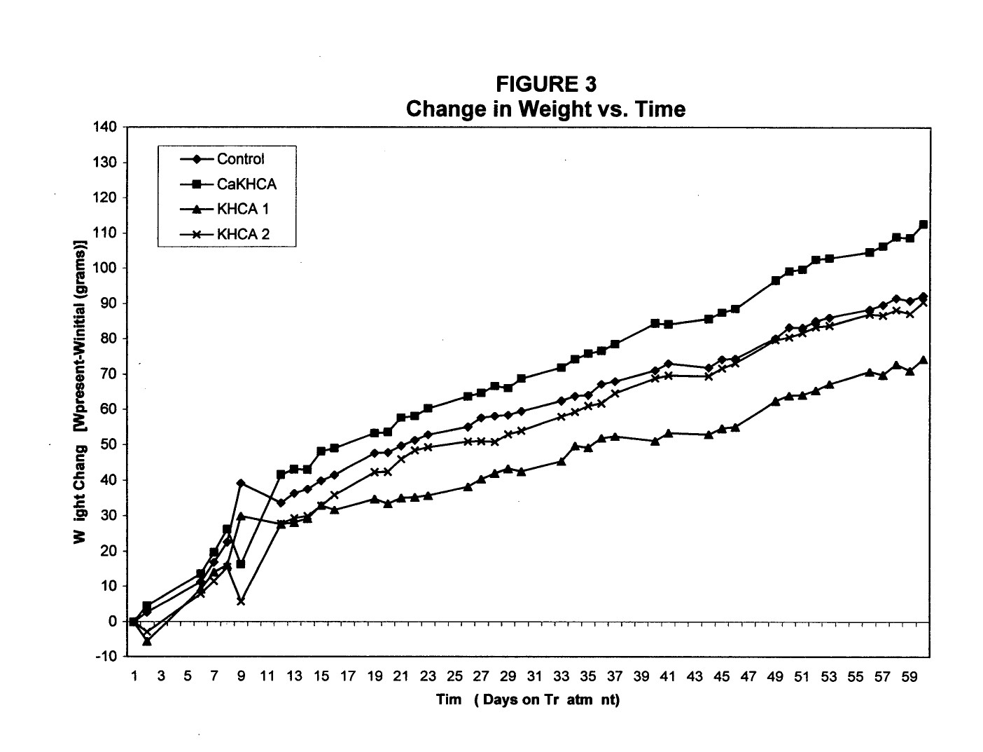

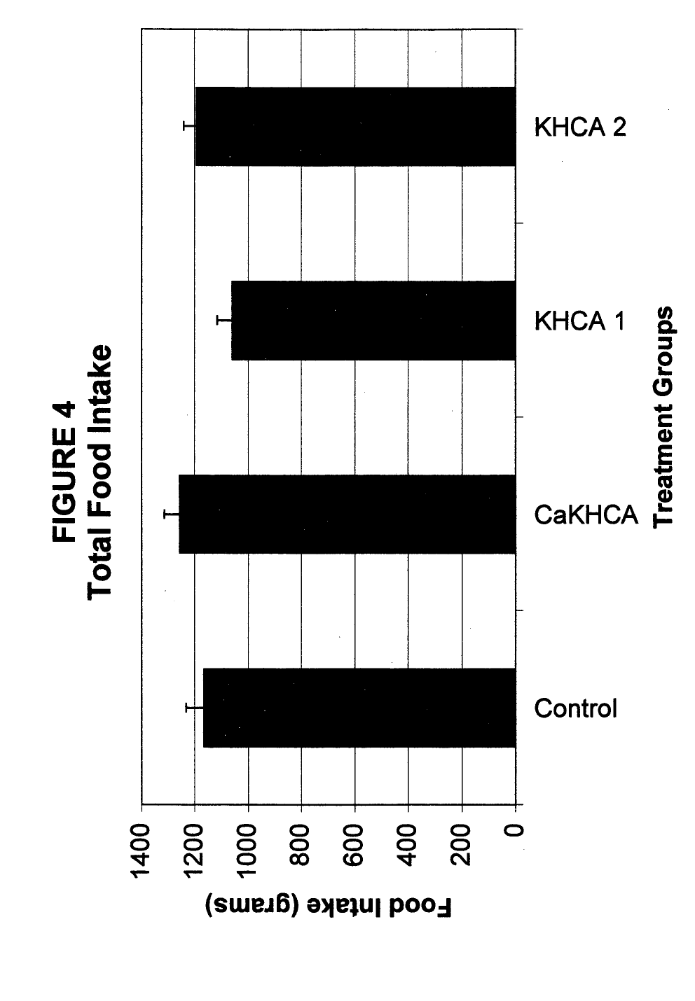

FIGS. 2 and 3 show the changes in average body weights in each arm over time and the average number of grams gained in weight in each arm over time. As can be seen from FIG. 2, the KHCA 1 group using the purer potassium salt began at an average weight almost the same as that of control, but diverged dramatically after about 9 days and remained strikingly lighter than control for the rest of the trial despite the convergence in food intakes at the end of the period. In contrast, the CaKHCA group increased its body weight vis-á-vis control from day 9 onward. The KHCA 2 group ingesting the poor quality potassium salt began as the heaviest of the arms (about 24 grams heavier than control) and ended the trial still about 24 grams, on average, heavier than control. FIG. 3 makes these points clearer. The lines showing the number of grams gained over time indicate quite directly that the CaKHCA salt group underwent significant weight gain in relation to control, the KHCA 2 group gained weight at a rate only slightly less than was true of control, and the KHCA 1 group obviously gained much less weight than did control. Below is the food intake data for the three active arms compared to that of control over the 60 days.

TOTAL FOOD INTAKE

| Total Food | |
|---|---|
| Intake (grams) | |
| | Mean |
| Control | 1165.9 |
| CaKHCA | 1258.4 |
| KHCA 1 | 1060.1 |
| KHCA 2 | 1194.1 |
| | Stdev |
| Control | 152.0 |
| CaKHCA | 126.7 |
| KHCA 1 | 127.5 |
| KHCA 2 | 109.0 |
| | Avg FI |
| Control | 1238.98 |
| CaKHCA | 1320.44 |
| KHCA 1 | 1144.86 |
| KHCA 2 | 1275.30 |

This material supports the two biphasic attributes suggested here for HCA. In agreement with other researchers, the inventor has found that the appetite suppression of HCA does not appear to last for more than seven weeks in the rat model in normal animals. By day 60, any anorectic effect had disappeared in the study outlined here.

The biphasic dose response issue on a diet supplying a nontrivial percentage of its calories as fat apparently has not been explored before. In this study, the salts supplied to all three active arms contained the same amounts of HCA. Strikingly, the apparently lower availability of HCA for physiologic uptake or usage when delivered in the form of CaKHCA emerged despite the widespread assertion among commercial suppliers of HCA products that issues of bioavailability are adequately addressed simply by making the calcium salt of the compound soluble. Such is not the case. Similarly, the lower quality potassium salt, KHCA 2, in which inadequate amounts of potassium were available to fully occupy all bonding sites, proved to be no better, but also no worse, than placebo as a weight loss agent. Only the relatively clean and relatively fully reacted KHCA 1 showed any anorectic effect upon food consumption and weight gain in this model. The negative findings with the CaKHCA arm offer proof that a particular usage or dosage of HCA will increase the activity of acetyl CoA carboxylase, and, depending upon the dose and the diet, lead either to a null result or to a gain in appetite and weight. As can be seen in the following chart, at the level of intake used experimentally on a 30% fat diet, potassium HCA tends to increase protein as a percentage of body weight while reducing fat as a percentage of body weight. The relatively higher rates of body hydration found in the potassium salt-fed arms may represent elevated glycogen stores in muscle, an expected finding.

| Mean | Control | CaKHCA | KHCA 1 | KHCA 2 |
|---|---|---|---|---|
| % Body H₂O | 56.70 | 56.06 | 59.96 | 58.93 |
| % Protein | 18.66 | 17.77 | 18.95 | 20.07 |
| % Fat | 20.42 | 22.56 | 17.83 | 18.27 |
| % Ash | 2.98 | 2.37 | 3.04 | 2.61 |

The data from this experiment clearly indicate that there is a level of intake at which HCA increases both appetite and weight gain, points obviously beneficial in cachexia and excess catabolism.

### EXAMPLE 2

### Insulin, Leptin & Glucocorticoids

One could hardly suggest that HCA might be used to control cachexia, excess catabolism, or sarcopenia if the compound contributed to the further dysregulation of hormones already dysregulated in these conditions. Therefore, it is important to know that HCA does not lead to hormonal imbalances. Data was collected from the rat study described above with regard to serum insulin, leptin and cortisol levels to establish the effects upon these hormones.

| | Insulin | Leptin | Corticosterone |
|---|---|---|---|
| Group | ng/mL | ng/mL | ng/mL |
| Control | 2.655 | 9.52 | 269.38 |
| Control | 7.077 | 18.94 | 497.87 |
| Control | 4.280 | 34.34 | 265.71 |
| Control | 9.425 | 24.32 | 209.54 |
| Control | 3.798 | 8.40 | 116.12 |
| KHCA 1 | 3.880 | 9.93 | 45.79 |
| KHCA 1 | 4.399 | 7.31 | 33.10 |
| KHCA 1 | 3.181 | 9.25 | 65.57 |
| KHCA 1 | 3.210 | 24.36 | 55.40 |
| KHCA 1 | 3.639 | 9.07 | 84.62 |
| KHCA 2 | 4.427 | 9.13 | 26.02 |
| KHCA 2 | 4.301 | 9.75 | 270.83 |
| KHCA 2 | 3.245 | 8.00 | 45.44 |
| KHCA 2 | 3.695 | 9.16 | 45.63 |
| KHCA 2 | 2.053 | 8.26 | 38.04 |

Both of the potassium (−)-hydroxycitrate arms were superior to the calcium/potassium arm (data not shown here) in reducing insulin, leptin and corticosterone concentrations. Because of the difficulty in achieving significance with only 5 data points per arm, calculations regarding insulin and leptin combined the data from the two KHCA arms. With respect to insulin, the one-tailed P value was a significant 0.0306, and the two-tailed P value fell slightly short of significance at 0.0612. Using this combined data, there was also a significant one-tailed P value difference between the two KHCA arms and the result found with the CaKHCA. With respect to leptin, the two KHCA arms were combined, in part, because of one anomalously high data point and yielded a one-tailed P value which was a significant 0.0241 and a two-tailed P value which was significant at 0.0482. Corticosterone results were highly significant even at 5 data points per arm. KHCA 1 was easily significantly superior to control: the one-tailed P value was a highly significant 0.0048, and the two-tailed P value was a highly significant 0.0096.

Non-esterified fatty acid levels were not significantly different between control and the KHCA arms, but serum glucose and triglyceride levels exhibited a trend towards elevation. This is consistent with HCA's biophasic properties on a fatty diet and with published animal data to the effect that HCA elevates fatty acid oxidation at rest. Elevated fatty acid oxidation typically slightly increases some fractions of blood fats, and also increases the rate of gluconeogenesis, hence may slightly increase blood glucose levels. However, in those individuals with markedly elevated blood glucose levels/glucose dysregulation, HCA can be used to improve glucose regulation. (U.S. Pat. No. 6,207,714) The same has been shown in animals with regard to elevated blood fats, in which case these blood fats are reduced.

The clear implication of these data is that HCA may be useful in reducing insulin levels and insulin resistance, elevated leptin levels and leptin resistance, and elevated glucocorticoid levels. Inasmuch as counterregulatory hormones, such as cortisol, and supposedly anabolic hormones, such as insulin, may both play roles in cachexia and other forms of unwanted weight loss and, likewise, tend strongly to become dysregulated with advancing years, these findings indicate the safety of HCA and other possible mechanisms of action in the proposed use.

### EXAMPLE 3

Numerous methods can be given as means of delivering HCA as required by the invention, including capsules, tablets, powders and liquid drinks. The following preparation will provide a stable and convenient dosage form.

| Ingredient | | Weight | Percent | 1 Kg Batch |
|---|---|---|---|---|
| 1. | Aqueous Potassium | 500 gm | 62.5% | 0.63 |
| | Hydroxycitrate | | | |
| 2. | Calcium Carbonate | 50 gm | 6.25% | 0.06 |
| 3. | Potassium Carbonate | 50 gm | 6.25% | 0.06 |
| 4. | Anhydrous Lactose | 150 gm | 18.75% | 0.19 |
| 5. | Cellulose Acetate | 50 gm | 6.25% | 0.06 |
| | Pthalate Acetate | | | |
| | Total | 800 gm | 100.00% | 100.00 |

A. Blend items 1-5 in mixing bowl until smooth and even.
B. Take the liquid and spray into spray-drying oven at 300° C. until white powder forms. When powder has formed, blend with suitable bulking agent, if necessary, and compress into 800 mg tablets with hardness of 10-15 kg. This will mean that each tablet, if starting with 62% KHCA polymer powder, will have about 31% KHCA. However, if the tablets are pressed to 1600 mg, the dose will be equal to 800×62% KHCA.
C. After pressing the granulate through the screen, make sure that it flows well and compress into oblong tablets.
D. Tablets should have a weight of 1600 mg and a hardness of 14±3 kg fracture force. When tablets are completed, check for disintegration in pH 6.8, 0.05M KH2PO4. Disintegration should occur slowly over 4-5 hours.

### CONCLUSION

(−)-Hydroxycitrate has a multitude of metabolic functions. The literature teaches that the compound reduces blood lipids, induces weight loss and decreases appetite in both animals and humans. However, the inventor has discovered an entirely novel use, to wit, for treating and ameliorating cachexia, health-threatening catabolism and unhealthful weight loss, such as in sarcopenia. This is accomplished without disturbing hormonal and central mechanisms of metabolic regulation.

1. A method for treating or ameliorating cachexia, health-threatening catabolism and unhealthful weight loss in an individual in need thereof which is comprised of administering orally an effective amount of (−)-hydroxycitric acid.

2. The method of claim 1 where the (−)-hydroxycitric acid is supplied as a therapeutically effective amount of the free acid or its lactone.

3. The method of claim 1 where the (−)-hydroxycitric acid is supplied as a therapeutically effective amount of the alkali metal salts potassium or sodium (−)-hydroxycitrate.

4. The method of claim 1 where the (−)-hydroxycitric acid is supplied as a therapeutically effective amount of the alkaline earth metal salts calcium or magnesium (−)-hydroxycitrate.

5. The method of claim 1 where the (−)-hydroxycitric acid is supplied as a therapeutically effective amount of a mixture the alkali metal salts and/or the alkaline earth metal salts of(−)-hydroxycitrate or some mixture of alkali metal salts and alkaline earth metal salts of (−)-hydroxycitrate or in the form of therapeutically effective amide and/or ester derivatives of (−)-hydroxycitric acid.

6. The method of claim 1 where the (−)-hydroxycitric acid is supplied in a therapeutically effective amount as the free acid, its lactone or as one or more of the salts or other derivatives of the free acid and is delivered in a controlled release form.
